(19) United States
(12) Patent Application Publication
(10) Pub. No.: US 2002/0132823 A1
(43) Pub. Date: Sep. 19, 2002

(54) Assay method

(76) Inventors: Jiahuai Han, San Diego, CA (US); Liguo New, San Diego, CA (US); Hermann Gram, Weil (DE)

Correspondence Address: THE SCRIPPS RESEARCH INSTITUTE, OFFICE OF PATENT COUNSEL, TPC-8, 10550 NORTH TORREY PINES ROAD, LA JOLLA, CA, 92037, US

(21) Appl. No.: 09/765,206

(22) Filed: Jan. 17, 2001

(51) Int. Cl. A61K 31/506; C 07D 4 3/14

(52) U.S. Cl. 514/275; 544/324

(57) ABSTRACT

An assay is provided for the identification and comparison of compounds having activity as modulators of the expression and release of TNFa, IL-1 or IL-6 in humans and animals in which modulation of the catalytic activity of PRAK (p38-regulated/activated protein kinase) is employed for identification and comparison of test compounds.

### GOVERNMENT RIGHTS

This invention was made with government support under Grant GM51417, AI41637 and AHA95007690 awarded by the National Institutes of Health. The U.S. Government has certain rights in the invention.

### FIELD OF THE INVENTION

This invention relates to biologically active compounds, in particular to compounds which have activity as modulators of the expression and release of TNFa, IL-1 or IL-6 in humans and animals, to processes for the identification of such compounds and also to the therapeutic use of such compounds for the treatment of TNFa, IL-1 or IL-6 mediated diseases such as rheumatoid arthritis and diseases of bone metabolism, e.g. osteoporosis.

### BACKGROUND OF THE INVENTION

PRAK (p38-regulated/activated protein kinase) is a recently discovered protein kinase which is regulated by p38 MAP kinase, and was first described by New et al. (New L., Jiang Y., Zhao M., Liu K., Zhu W., Flood L. J., Kato Y., Parry G. C. N. and Han J.; EMBO Journal, Vol. 17, No. 12, pp. 3372-3384, 1998). New et al. report that activated PRAK is able to phosphorylate small heat shock protein 27 (HSP27) at the physiologically relevant sites, but indicate that the question of whether PRAK is a physiologically relevant kinase for small heat shock proteins awaits further investigation.

We have now found that small molecular weight inhibitors of the catalytic activity of PRAK in vitro inhibit the production of the inflammatory mediators TNFa, IL-1 or IL-6.

For ease of reference, compounds which have activity as modulators of the expression and release of TNFa, IL-1 or IL-6 in humans and animals are hereinafter referred to as TNF Modulators.

### DESCRIPTION OF THE INVENTION

Accordingly in a first aspect the invention provides use of PRAK for the identification of a TNF Modulator.

In particular, the invention may be used for the identification of TNF Modulators which are inhibitors of PRAK.

Thus in a preferred embodiment the first aspect of the invention provides a method for the identification of a TNF Modulator which comprises contacting a test compound with a system comprising a PRAK protein and monitoring the system for inhibition of PRAK catalytic activity.

For the purposes of the present description “PRAK protein” means a full length human or animal PRAK, e.g. the 471 amino acid human PRAK protein described by New et al. (ibid), or any fragment or analogue thereof having PRAK catalytic activity.

The method of the invention (hereinafter the PRAK Inhibition assay) may be used for screening of individual compounds and for screening of compound collections for TNF Modulators; for example for screening of compound collections or libraries comprises from a few compounds up to tens of thousands of compounds or more, including combinatorial compound libraries. The Prak inhibition assay may be used as a first line screening assay to identify lead compounds. Alternatively a similar assay may be used to compare and quantify the PRAK inhibition activity of compounds, e.g. to compare compounds produced from medicinal chemistry lead optimization/derivatisation programmes.

Thus in a further aspect the invention provides a method for the comparison of TNF Modulators, comprising separately contacting the TNF Modulators with a system comprising a Prak protein and comparing each system for inhibition of PRAK catalytic activity (hereinafter referred to as the PRAK Comparison assay).

The compounds for screening, e.g. primary screening, may be synthetic chemical compounds or naturally occurring compounds or compounds extracted from nature or from culture systems, or mixtures of any of these.

Typically the PRAK protein is activated prior to use in the PRAK Inhibition or PRAK Comparison assay. For instance, the PRAK protein is phosphorylated by incubation with an appropriate phosphorylating enzyme, e.g. phospho-p38; for example as hereinafter described in the Example.

The activated PRAK protein is conveniently contacted with test compound in solution. For example, the activated PRAK protein may be prepared in an appropriate buffer, e.g. kinase buffer, and the test compounds dissolved in an appropriate solvent, e.g. DMSO, and the two solutions mixed together with other reagents as required.

Any suitable methodology may be used to monitor for inhibition of PRAK catalytic activity, conveniently by monitoring the absence or decrease in PRAK catalytic activity in the presence of test compound as compared with the absence of test compound. Thus, the catalytic activity of PRAK protein on one or more substrates may be used to indicate PRAK catalytic activity and a complete or partial inhibition of this activity may be used to indicate PRAK inhibition. Preferably the activity of PRAK protein as a kinase for phosphorylation of heat shock protein, e.g. HSP27, may be used to monitor the outcome of the PRAK Inhibition or PRAK Comparison assay.

Thus for example, activated PRAK protein, heat shock protein, e.g. HSP27, and phosphate donor, e.g. ATP, are incubated together in the presence of test compound and the extent of phosphorylation of heat shock protein which results is compared with that which results in the absence of test compound. A complete or partial inhibition of phosphorylation of heat shock protein indicates that the test compound is a TNF Modulator as defined above. Conveniently the extent of phosphorylation of heat shock protein may be determined using appropriate labeling, e.g. labeling of the phosphate donor substrate, e.g. conveniently by use of γ³³P-ATP. The amount of labeled material present in the phosphorylated heat shock protein when the assay is conducted in the presence of test compound, as compared with when the assay is conducted in the absence of test compound, is indicative of the inhibitory activity of the test compound.

In a further aspect the invention includes TNF Modulators when identified by the PRAK Inhibition assay or when selected by a method comprising the the PRAK Comparison assay, hereinafter referred to as TNF Modulators of the Invention.

In particular TNF Modulators of the Invention possess PRAK inhibiting activity and thus are indicated as inhibitors of production of inflammatory cytokines, such as TNF-a IL-1 and IL-6, and also to potentially block the effects of these cytokines on their target cells. These and other pharmacological activities of the Agents of the Invention as may be demonstrated in standard test methods for example as described below in the Example.

TNF Modulators of the Invention typically have IC₅₀s for PRAK inhibition of about 1 μM or less, e.g. from about 10 μM to about 100 nM or less, e.g. of about 10 nM or less.

As indicated in the Example TNF Modulators of the Invention are potent inhibitors of TNF-a release. Accordingly, the TNF Modulators of the Invention have pharmaceutical utility as follows:

TNF Modulators of the Invention are useful for the prophylaxis and treatment of diseases or pathological conditions mediated by cytokines such as TNFa, IL-1 or IL-6 e.g., inflammatory conditions, autoimmune diseases, severe infections, and organ or tissue transplant rejection, e.g. for the treatment of recipients of heart, lung, combined heartlung, liver, kidney, pancreatic, skin or corneal transplants and for the prevention of graftversushost disease, such as following bone marrow transplants.

TNF Modulators of the Invention are particularly useful for the treatment, prevention, or amelioration of autoimmune disease and of inflammatory conditions, in particular inflammatory conditions with an aetiology including an autoimmune component such as arthritis (for example rheumatoid arthritis, arthritis chronica progrediente and arthritis deformans) and rheumatic diseases. Specific autoimmune diseases for which Agents of the Invention may be employed include autoimmune haematological disorders (including e.g. hemolytic anaemia, aplastic anaemia, pure red cell anaemia and idiopathic thrombocytopenia), systemic lupus erythematosus, polychondritis, sclerodoma, Wegener granulamatosis, dermatomyositis, chronic active hepatitis, myasthenia gravis, psoriasis, StevenJohnson syndrome, idiopathic sprue, autoimmune inflammatory bowel disease (including e.g. ulcerative colitis and Crohn's disease), endocrine ophthalmopathy, Graves disease, sarcoidosis, multiple sclerosis, primary biliary cirrhosis, juvenile diabetes (diabetes mellitus type I), uveitis (anterior and posterior), keratoconjunctivitis sicca and vernal keratoconjunctivitis, interstitial lung fibrosis, psoriatic arthritis and glomerulonephritis (with and without nephrotic syndrome, e.g. including idiopathic nephrotic syndrome or minimal change nephropathy).

TNF Modulators of the Invention are also useful for the treatment, prevention, or amelioration of asthma, bronchitis, pneumoconiosis, pulmonary emphysema, and other obstructive or inflammatory diseases of the airways.

TNF Modulators of the Invention are useful for treating undesirable acute and hyperacute inflammatory reactions which are mediated by TNF, especially by TNFa, e.g., acute infections, for example septic shock (e.g., endotoxic shock and adult respiratory distress syndrome), meningitis, pneumonia; and severe burns; and for the treatment of cachexia or wasting syndrome associated with morbid TNF release, consequent to infection, cancer, or organ dysfunction, especially AIDS—related cachexia, e.g., associated with or consequential to HIV infection.

TNF Modulators of the Invention are particularly useful for treating diseases of bone metabolism including osteoarthritis, osteoporosis and other inflammatory arthritides. For the above indications the appropriate dosage will, of course, vary depending, for example, on the particular TNF Modulator of the Invention employed, its activity, the subject to be treated, the mode of administration and the nature and severity of the condition being treated.

The TNF Modulators of the Invention may be administered by any conventional route, e.g. orally, for example in the form of solutions for drinking, tablets or capsules or parenterally, for example in the form of injectable solutions or suspensions. Normally for systemic administration oral dosage forms are preferred, although for some indications the TNF Modulators of the Invention may also be administered topically or dermally, e.g. in the form of a dermal cream or gel or like preparation or, for the purposes of application to the eye, in the form of an ocular cream, gel or eyedrop preparation; or may be administered by inhalation, e.g., for treating asthma.

The invention includes the use of the TNF Modulators of the Invention for the prophylaxis and treatment of diseases or pathological conditions mediated by cytokines such as TNFa, IL-1 or IL-6.

The invention includes:

i) A method of inhibiting production of soluble TNF, especially TNFa, or of reducing inflammation in a subject (i.e., a mammal, especially a human) in need of such treatment which method comprises administering to said subject an effective amount of a TNF Modulator of the Invention, or a method of treating any of the above mentioned conditions, particularly a method of treating an inflammatory or autoimmune disease or condition, e.g. rheumatoid arthritis, or alleviating one or more symptoms of any of the above mentioned conditions.

ii) A TNF Modulator of the Invention for use as a pharmaceutical, e.g. for use as an immunosuppressant or antiinflammatory agent or for use in the prevention, amelioration or treatment of any disease or condition as described above, e.g., an autoimmune or inflammatory disease or condition.

iii) A pharmaceutical composition comprising a TNF Modulator of the Invention in association with a pharmaceutically acceptable diluent or carrier, e.g., for use as an immunosuppressant or anti-inflammatory agent or for use in the prevention, amelioration or treatment of any disease or condition as described above, e.g., an autoimmune or inflammatory disease or condition.

iv) Use of a TNF Modulator of the Invention in the manufacture of a medicament for use as an immunosuppressant or anti-inflammatory agent or for use in the prevention, amelioration or treatment of any disease or condition as described above, e.g., an autoimmune of inflammatory disease or condition.

In a yet further aspect the invention includes TNF Modulators which are specific inhibitors of PRAK (hereinafter referred to as Specific TNF Modulators) and to the use of such Specific TNF Modulators for the prophylaxis and treatment of diseases or pathological conditions mediated by cytokines such as TNFa, IL-1 or IL-6; for instance, in methods, uses and compositions similar to those defined above in paragraphs i) to iv) for the TNF Modulators of the Invention.

For the purposes of the present description a Specific TNF Modulator is a compound which specifically inhibits PRAK as compared with other kinases of the p38 MAP kinase cascade. Conveniently Specific TNF Modulators inhibit PRAK with an IC₅₀ which is at least about 1 μM, e.g. from about 10 μM to about 100 nM or less, e.g. of about 10 nM or less, and have IC₅₀s for inhibition of other kinases of the p38 MAP kinase cascade, e.g. for inhibition of p38 MAP kinase, which are at least 10 μM, preferably at least 100 μM or more.

The invention is further described by way of illustration only in the following Example which relates to assay for in vitro PRAK kinase activity, and TNFα, IL-1β, and IL-6 activity and our finding that small molecular weight inhibitors of the catalytic activity of PRAK inhibit the product of TNFα, IL-1β, and IL-6 in vitro.

### EXAMPLE

Inhibitors of PRAK, a Downstream Kinase of p38a, have Anti-inflammatory Properties.

Prak was recently identified as a substrate of p38a (New et al. ibid). Prak is a kinase homologous to MapkapK2, MapkapK3, Mnk1 and Mnk2, and is able to efficiently phosphorylate the small heat shock protein HSP27 in vitro (New et al. ibid). Further in vivo substrates have not been identified yet, and the biological function of Prak is unknown to date.

Below we provide evidence that Prak is involved in the expression of TNFα, IL-1 or IL-6 in human peripheral blood mononuclear cells or dermal fibroblasts, based on the fact that small molecular weight inhibitors of the catalytic activity of Prak inhibit the production of the inflammatory mediators TNFa, IL-1, or IL-6 in vitro. Thus, Prak is apparently involved in the inflammatory response in vivo, and inhibition of this kinase provides a new anti-inflammatory principle.

Materials and Methods

1. In vitro Kinase Assay

Recombinant human Prak was expressed in E. coli as His6-tagged protein substantially as described for Mnk1 by Tschopp et al. (Tschopp et al. (2000) Phoshorylation of eIF-4E on Ser 209 in response to mitogenic and inflammatory stimuli is faithfully detected by specific antibodies. Molecular Cell Biology Res. Comm. 3, 205-211). Phospho-p38a and His6-Hsp27 was prepared substantially as described (New et al. and Tschopp et al. ibid). All kinase reactions were performed with the following reaction buffer. Kinase buffer (5×): 125 mM Hepes (Stock at 1M; Gibco #15630-056), 125 mM β-glycerophosphate (Sigma #G-6251):125 mM MgCl₂ (Merck #5833); 0.5 mM Sodium orthovanadate (Sigma #5-6508), 10 mM DTT (Boehringer Mannheim #708992), pH 7.4. The (5×) kinase buffer is freshly prepared from 25× stock solution. DTT is stored frozen at −20° C. in small aliquots and added immediately before use.

Prior to the assay, recombinant Prak is phosphorylated by incubation with phospho-p38. The kinase mix is prepared by mixing phospho-p38 (final concentration: 30 μg/ml) and Prak (final concentration: 150 μg/ml) in 1× kinase buffer (containing 5 μM ATP. The reaction mixture is incubated for 30 min at RT and chilled on ice until further use.

For the assay, the activated kinases are prediluted in 1× kinase buffer/0.00025% TWEEN20 to 15 μg/ml.

The compounds to be tested are prediluted in 10% DMSO to 10× the final assay concentration.

The substrate mix is prepared by mixing His-HSP27 (71.5 μg/ml) with ATP (1.43 μM) and ³^{₃₃}P-ATP (7.15 μCi/ml) in 1× kinase buffer/0.00025% TWEEN20. To this mix, 5 μl of compound are added, and the reaction is started by addition of 10 μl of kinase mix.

The assay is stopped after one hour of incubation at room temperature by the addition of 5 μl EDTA 0.5M. The label incorporated into protein is separated from labelled ATP by filtration through an Immobilon-P menbrane. After washing and drying the membrane, incorporated label is quantified by liquid scintillation counting. Percent inhibition is calculated relative to control reactions which do not contain inhibitor.

2. TNFα and IL-1 Release from Human PBMCs

Cell Separation and Culture

All cell culture and ELISA work was done on 96 well plates (Costar). Mononuclear cells were prepared from the blood of healthy volunteers using ficoll-hypaque density separation according to the method of Hansel et al (Hansel TT et al. (1991) An improved immunomagnetic procedure for the isolation of highly purified human blood eosinophils. J. Imm. Methods 145, 105-110) and used at a concentration of 100,000 cells/well in RPMI 1640, 5% FCS (Gibco). Serial dilution of the test compound were made in DMSO and diluted with culture medium. Final DMSO concentration in the culture medium was 0.1%. Compound were preincubated with the cells for 30 minutes at 37° C., 5% CO₂. Then, the cells were stimulated with LPS (5 μg/ml, Sigma) and γ-interferon (100 U/ml, Boehringer Mannheim) and cultured for 3 hours (for TNFα-determination) or 4.5 hours (for Il-1 β and Il-6 determination). The plates were centrifuged, the supernatants were removed and stored at −80° C. until ELISA determination.

TNFα-ELISA

For ELISA determination, supernatants were diluted 1:1 and added into a plate coated with the anti humanTNF monoclonal antibody (357-101-4, clone 92030603 from European Collection of Animal Cell Cultures, antibody produced and purified within Novartis Pharma, 1 μg/ml in PBS) After overnight incubation, the plates were washed (4×) and biotinylated anti humanTNF monoclonal antibody 2-179-E11 (clone 92030602 (European Collection of Animal Cell Cultures), produced and purified in Novartis Pharma) was added to a final concentration of 1 μg/ml (100 μl/well). After 4 hours of incubation, plates were washed 4× and streptavidin-alkaline phosphatase (Jackson Immunoresearch, #016-050-084, 1:7500 dilution) was added. The p-nitrophenyl phosphate substrate was prepared immediately before the assay from tablets (1 mg/ml in buffer pH 9.8, 100 μl/well). Color formation was monitored in a Bio-Tec ELx 808 plate reader at 405 nm in the kinetic mode for 30 minutes. A set of humanTNF standards (31.1 pg-2000 pg) is included on each plate and used to calculate TNF concentrations from the slopes for each individual well. IC₅₀-values were calculated using the Origin software package by weighted fitting of the means of percent inhibition for each inhibitor concentration to the logistic function.

Hu IL-1² ELISA

ELISA microtiter plates were coated with a murine anti-human IL-1² MAb (Human IL-1² Cytoset (Biosource International Inc., #CHC1214), 100 μl at 1 μg/ml) in PBS 0.02% NaN₃ and incubated overnight at +4° C., The following day, microtiter plates were washed 4 times with PBS/0.05% Tween/0.02% NaN₃ and blocked with 300 μl of PBS/2% bovine serum albumin (BSA)/0.02% NaN₃ for 3 h. Plates were washed again (4 times) and 100 μl of supernatant (final dilutions of 1:15 and 1:30) or of the recombinant human IL-1² standard (titration curve ranging from 640 to 10 pg/ml in 2 fold dilution steps) was added in duplicate together with a biotin-labelled murine anti-human IL-1² MAb (50¼ l at 0.4 μg/ml). The plates were incubated for 2 h at room temperature with shaking (700 rpm). After incubation the plates were washed 4 times, and streptavidin alkaline phosphatase conjugate (Jackson Immunoresearch, #016-050-084) was added at a final dilution of 1/3000 (100 ¼ l/well; 30 min at room temperature). After washing (4 times) the substrate (p-nitrophenylphosphate in diethanolamine buffer; 100 μl ) was added for 30 min. Reaction was blocked by the addition of 50 μl/well of NaOH. Plates were read in a microtiter reader (Bio-Rad) using filters of 405 and 490 nm.

3. Cytotoxicicty

THP-1 cells are obtained from ATCC and stored in liquid nitrogen. Medium for growth of THP-1 cells: RPMI 1640(GIBCO)+10% fetal bovine serum (GIBCO)+L-glutamine (2 mM)+penicillin/streptomycin (100 U/ml) +β-mercaptoethanol (0.05 mM). Cell culture medium for THP-1 incubation: RPMI 1640(GIBCO)+5% fetal bovine serum (GIBCO)+L-glutamine (2 mM)+penicillin/streptomycin (100 U/ml)+β-mercaptoethanol (0.05 mM).

The solution for stimulation is prepared by mixing equal volumes of IFNγ (Boehringer Mannheim, 2000 U/ml) and LPS (Sigma, L8274, 100 μg/ml). The dye solution is prepared from 500 mg MTT (Sigma, M2128) in 100 ml phosphate buffered saline (PBS) and kept under sterile conditions at 4° C. Sodium dodecyl sulfate (SDS) solution (10%) is made from 50 g SDS (Bioprobe Systems) and 5 ml 1N HCl. The solution is made up to 500 ml with distilled water and kept at room temperature (precipitates in the fridge). All incubations are done at 37° C. in 5% CO₂.

Test compounds are dissolved in dimethylsulfoxide (DMSO) at 10 mM and further diluted in DMSO to 3 mM and 1 mM. The DMSO stock solutions are further diluted in RPMI 1640 to 300 μM, 100 μM and 30 μM. The final DMSO concentration is 0.1 or 0.3%.

The 96-well plates for the incubation are prepared as follows: In duplicate wells, 60 μl cell culture medium, 20 μl compound and 100 μl THP-1 cell culture (500,000 cells/ml) are pre-incubated for 30 min.

To stimulate the cells, 20 μl of the LPS/β-IFN solution is added and the cells are incubated for 24 hours.

At the end of the incubation period, the plates are centrifuged and 100 μl medium is removed. Dye solution (10 μl) is added to each well and the plates are incubated for 4 hours or overnight. SDS-solution (100 μl) is added to each well and the plates are again incubated for at least 10 hours.

The absorbance at 540 nm is read with a microplate reader (Biotec 808 Elx). Blue-colored wells indicate living cells, whereas yellow wells indicate a high percentage of dead cells.

Calculation of cytotoxicity: The absorbance corresponding to 100% cytotoxicity equals the absorbance of wells without cells (no MTT converted into the blue dye). The absorbance corresponding to no cytotoxicity is taken from wells without substance, but with control solvent. Percent cytotoxicity is calculated for each substance concentration.

4. IL-6 Production in Human Dermal Fibroblasts

Neutralization Assay

Human dermal foreskin fibroblasts were obtained from Clonetics (CC-2509) and grown in FBM (Clonetics, CC-3131 ) including bFGF (1 ng/ml, CC-4065), insulin (5 μg/ml, CC-4021), and 2% FCS (CC-4101).

For induction of IL-6, cells were seeded at a density of 10⁴ cells per well in a 48 well tissue cluster. The following day, cells were starved for 6-7 h in FBM containing 2% FCS. Compounds were added at final concentrations of 10 and 1 μM 30 min prior to stimulation with recombinant human IL-1β (100 pg/ml).

Cell supernatant was taken 16-17 h after stimulation and the amount of released IL-6 determined in a sandwich ELISA.

IL-6 ELISA

ELISA microtiter plates were coated with a murine anti-human IL-6 MAb (314-14 (Novartis Pharma; batch EN23,961, 5.5 mg/ml); 100 μl at 3 μg/ml) in PBS 0.02% NaN₃ and incubated overnight at +4° C., The following day, microtiter plates were washed 4 times with PBS/0.05% Tween/0.02% NaN₃ and blocked with 300 μl of PBS/3% bovine serum albumin (BSA)/0.02% NaN₃ for 3 h. Plates were washed again (4 times) and 100 μl of supernatant (final dilutions of 1:20) or of the recombinant human IL-6 standard ((Novartis Pharma #91902), titration curve ranging from 1 to 0.0156 ng/ml in 2 fold dilution steps) was added in duplicate. After an overnight incubation at RT the plates were washed (4 times) and a different murine anti-human IL-6 MAb (110-14, Novartis Pharma; 6.3 mg/ml); 100 ¼ l at 1 μg/ml; 3 h at room temperature) was added. After additional 4 washes, a biotin-labelled goat anti-mouse IgG2b antiserum (Southern Biotechnology; #1090-08) was added at the final dilution of 1/10000 (100 μl/well; 3 h at room temperature). After incubation plates were washed 4 times and streptavidin coupled to alkaline phosphatase (Jackson Immunoresearch, #016-050-084) was added at a final dilution of 1/3000 (100 ¼ l/well; 30 min at room temperature). After washing (4 times) the substrate (p-nitrophenylphosphate in diethanolamine buffer; 100 μl) was added for 30 min. Reaction was blocked by the addition of 50 μl/well of 1.5 M NaOH. Plates were read in a microtiter reader (Bio-Rad) using filters of 405 and 490 nm. IL-6 levels in culture supernatants were calculated in reference to the standard curve using the cubic curve fit. Percentage inhibitions and IC₅₀ values were calculated

Results

As shown in the Table, small molecular weight compounds which inhibit the kinase activity of Prak in vitro, inhibit with a similar IC50 the release of TNFa and IL-1b from LPS-treated human peripheral mononuclear cells. In addition, the production of IL-6 is inhibited in human dermal fibroblasts by these compounds.

| | In vitro | | | | |
|---|---|---|---|---|---|
| | kinase | | IL-1β | | Inhibition |
| | assay | TNFα | release | Cyto- | of IL-6 @ |
| | Prak | release | IC50 | toxicity | 1 μM |
| Compound | IC50 [nM] | IC50 [nM] | [nM] | IC50 [nM] | [%] |
| Compound | 1100 | 2200 | 1100 | >30000 | 100 |
| A | | | | | |

Compound A has the following structural formula

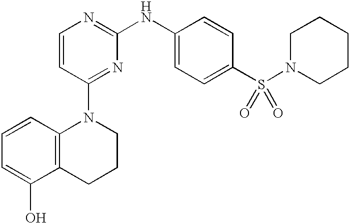

Compound A is included per se within the scope of the present invention.

1. Use of PRAK for the identification of a TNF Modulator.

2. Method for the identification of a TNF Modulator which comprises contacting a test compound with a system comprising a PRAK protein and monitoring the system for inhibition of PRAK catalytic activity.

3. Method for the comparison of TNF Modulators, comprising separately contacting the TNF Modulators with a system comprising a Prak and comparing each system for inhibition of PRAK catalytic activity.

4. A TNF Modulator when identified by a method according to claim 2.

5. A TNF Modulator when selected by a method comprising the method of claim 3.

6. The use of a TNF Modulator according to claim 4 or 5 for the prophylaxis and treatment of diseases or pathological conditions mediated by cytokines such as TNFa, IL-1 or IL-6.

7. i) A method of reducing inflammation in a subject in need of such treatment which method comprises administering to said subject an effective amount of a TNF Modulator according to claim 4 or 5;
ii) A TNF Modulator according to claim 4 or 5 for use as a pharmaceutical;
iii) A pharmaceutical composition comprising a TNF Modulator according to claim 4 or 5 in association with a pharmaceutically acceptable diluent or carrier, or
iv) Use of a TNF Modulator according to claim 4 or 5 in the manufacture of a medicament for use as an immunosuppressant or anti-inflammatory.

8. A TNF Modulator which is a specific inhibitor of PRAK.

9. The use of a TNF Modulator which is a specific inhibitor of PRAK for the prophylaxis and treatment of diseases or pathological conditions mediated by cytokines such as TNFa, IL-1 or IL-6.

10. The compound having the structural formula [chemical structure]
